(19) United States
(12) Patent Application Publication
(10) Pub. No.: US 2023/0256002 A1
(43) Pub. Date: Aug. 17, 2023

(54) Hard lozenge made with dietary fiber

(71) Applicants: Keith Allen Garleb, Westerville, OH (US); Kevin Barry Garleb, Westerville, OH (US)

(72) Inventors: Keith Allen Garleb, Westerville, OH (US); Kevin Barry Garleb, Westerville, OH (US)

(21) Appl. No.: 18/164,837

(22) Filed: Feb. 6, 2023

Related U.S. Application Data
(60) Provisional application No. 63/267,854, filed on Feb. 11, 2022.

(51) Int. Cl. A61K 31/716; A61K 9/00; A61K 31/702; A61K 47/26

(52) U.S. Cl. CPC A61K 31/716; A61K 9/0056; A61K 31/702; A61K 47/26

(57) ABSTRACT

Disclosed are compositions of dietary fiber and nonnutritive bulk sweeteners and/or nonnutritive high-intensity sweeteners and/or flavors in a hard, amorphous structure that can be administered to humans and dissolves slowly in the mouth and supports gastrointestinal tract health.

### CROSS-REFERENCE TO RELATED APPLICATIONS

This application relates to a prior provisional patent application. The application number of the prior provisional patent application is 63/267,854 and it was filed on Feb. 11, 2022.

### BACKGROUND OF THE INVENTION

The present invention pertains to a food composition comprising a hard lozenge composed of dietary fiber. More particularly, the invention relates to dietary fiber and nonnutritive bulk sweeteners and/or nonnutritive high-intensity sweeteners and/or flavors that are combined to form a hard, amorphous structure that dissolves slowly in the mouth and supports gastrointestinal tract health.

People are encouraged to consume more dietary fiber. Dietary fiber is the edible parts of plants or analogous carbohydrates that are resistant to digestion and absorption in the human small intestine, with complete or partial fermentation in the large intestine. The adequate intake for dietary fiber is set at 14 grams per 1000 kilocalories. On average, adult men and women should be consuming 38 grams and 25 grams per day of dietary fiber, respectively. However, data from the National Health and Nutrition Examination Survey 2015-2016 show that dietary fiber intake in the United States averages only 17 grams per day for adults. Dietary fiber provides multiple health benefits related to areas such as cardiovascular disease, type 2 diabetes, cancer, and gastrointestinal health. (Dahl, W.; Stewart, M. Position of the Academy of Nutrition and Dietetics. Health Implications of Dietary Fiber. Journal of the Academy of Nutrition and Dietetics. 2015, 115, 1861-1870.) From a gastrointestinal tract health standpoint, dietary fiber helps to normalize bowel function and serves as an energy source for the gut microbiota. Gut microbiota use dietary fiber as an energy source through a process called anaerobic fermentation. During fermentation, the microbiota produces short chain fatty acids that provide numerous benefits such as stimulating water absorption (Binder, H. Role of Colonic Short-Chain-Fatty-Acid Transport in Diarrhea. Annual Review of Physiology. 2010, 72, 297-313), producing an environment in the large bowel that is not conducive to the growth of pathogenic organisms (Lawly, T.; Walker, A. Intestinal Colonization Resistance. Immunology. 2013, 138, 1-11), and serving as an energy source for the colonic mucosa. (Roediger, W. Utilization of Nutrients by Isolated Epithelial Cells of the Rat Colon. Gastroenterology. 1982, 83, 424-429.)

There is a need for a novel, low calorie product that delivers dietary fiber in a palatable and convenient form to the consumer.

### SUMMARY OF THE INVENTION

The objective of the invention is to provide a hard lozenge consisting of dietary fiber. According to the first embodiment of the invention, a food composition is provided that comprises a hard lozenge that is composed of a blend of dietary fibers and nonnutritive bulk sweeteners and/or nonnutritive high-intensity sweeteners and/or flavorings. According to the second embodiment of the invention, a hard lozenge is provided that comprises a blend of dietary fibers and nonnutritive bulk sweeteners and/or nonnutritive high-intensity sweeteners and/or flavorings in a hard, amorphous structure that dissolves slowly in the mouth and supports gastrointestinal tract health.

### DETAILED DESCRIPTION OF THE INVENTION

A hard lozenge comprised of a blend of dietary fibers and a nonnutritive bulk sweetener and/or nonnutritive high-intensity sweeteners and/or flavorings is discussed herein. The present disclosure is considered to exemplify the invention and is not intended to limit the invention to the specific embodiments illustrated by the descriptions below. The term “dietary fiber” as used herein, unless otherwise specified, refers to the edible parts of plants or analogous carbohydrates that are resistant to digestion and absorption in the human small intestine, with complete or partial fermentation in the large intestine. The term “nondigestible” as used herein, unless otherwise specified, refers to material not being digested and absorbed in the human small intestine. The term “lozenge” as used herein, unless otherwise specified, refers to a hard, amorphous structure that dissolves slowly in the mouth. The term “amorphous” as used herein, unless otherwise specified, refers to a state of material where the molecules have a random arrangement. The term “glucose polymer” as used herein, unless otherwise specified, refers to large molecules composed of greater than five glucose molecules. The term “fructooligosaccharides” as used herein, unless otherwise specified, refers to a linear chain of fructose molecules, linked by beta (2-1) bonds ranging from 2 to 5 units and often terminate in glucose. The term “nonnutritive” as used herein, unless otherwise specified, refers to not providing nourishment by delivering little to no calories to the diet. The term “sweetener” as used herein, unless otherwise specified, is a substance that imparts sweetness. The term “flavoring” as used herein, unless otherwise specified, refers to a substance that provides flavor. The term “nonnutritive bulk sweetener” as used herein, unless otherwise specified, refers to an ingredient that does not provide nourishment by delivering little to no calories to the diet but provides bulk and sweetness to food comparable to that provided by sucrose and fructose. The term “nonnutritive high-intensity sweeteners” as used herein, unless otherwise specified, refers to an ingredient that does not provide nourishment by delivering little to no calories to the diet and provides essentially no bulk to food due to having a sweetness level greater than 100 times that of sucrose.

For the purpose of this invention, the sources of dietary fiber will include nondigestible fructooligosaccharides and nondigestible glucose polymers. An example of a nondigestible fructooligosaccharide is short-chain fructooligosaccharides. Short-chain fructooligosaccharides are composed of GF2 (1-kestose), GF3 (nystose), and GF4 (β-fructofuranosylnystose) with G representing glucose and F representing fructose. The degree of polymerization ranges from 3-5 monosaccharides with a molecular weight distribution of 500-830 Da. An example of a commercial source of short-chain fructooligosaccharides composed of GF2, GF3, and GF4 is Nutraflora P-95 (Ingredion Corporation, Westchester, Ill.). On a dry matter basis, Nutraflora P-95 is composed of 30-42% GF2, 45-57% GF3, and 5-15% GF4 (www.ingredion.com). According to specifications provided by Ingredion, Nutraflora P-95 is a powder with a maximum water content of 5%, a maximum sugar content (sucrose, glucose, fructose) of 5%, and a minimum of 95% short-chain fructooligosaccharides on a dry matter basis. Short-chain fructooligosaccharides cannot be digested by the endogenous enzymes of the human gastrointestinal tract and are considered a source of dietary fiber. An example of nondigestible glucose polymers includes resistant maltodextrins. Resistant maltodextrins are produced from starch with a process involving heat and/or acid and/or enzymes that form linkages that cannot be cleaved by mammalian enzymes in the gastrointestinal tract. An example of a resistant maltodextrin is Fibersol-2 (ADM/Matsutani, LLC, Decatur, Ill.). On a dry matter basis, the soluble fiber content of Fibersol-2 is 90% minimum. Fibersol-2 contains a maximum of 2% sugars (glucose, fructose, sucrose) on a dry matter basis. Fibersol-2 has a degree of esterification of less than 20 and an average molecular weight of 2,000 Da (Ohkuma, K.; Wakabayashi, S. Fibersol-2: a soluble, nondigestible, starch derived dietary fibre. Advanced Dietary Fibre Technology. 2017, 509-522.) For the purpose of this invention, the nonnutritive bulk sweetener will include the rare sugar D-allulose (Tate & Lyle, London, UK). D-allulose, also known as D-psicose, is a C-3 epimer of fructose that has about two-thirds the sweetness of sucrose but minimal caloric content (0.4 kcal/g). About 70% of D-allulose is absorbed in the small intestine within one hour but is excreted intact in the urine within 24 hours, while the other 30% is transported to the large intestine, where it is not fermented and thus excreted intact within 48 hours. The hard lozenge comprising dietary fiber will desirably include a nonnutritive high-intensity sweetener to provide the product with appealing sweetness. Examples of useful nonnutritive high-intensity sweeteners include stevia extract and monk fruit extract. The hard lozenge comprising dietary fiber will also desirably include flavoring and/or coloring to provide the product with an acceptable taste and appealing appearance for oral consumption. Examples of useful flavorings include peppermint, ginger, strawberry, mint, watermelon, cherry, lemon, orange, apple, grape, and raspberry.

The process to make the hard dietary fiber lozenge is similar to the process for making hard candy. In the process of making hard candy, sugar (sucrose), corn syrup, and water are combined and heated until reaching a temperature of approximately 300° F. (Hartel, R. Hard Candy: from hard crack to the glass transition. The Manufacturing Confectioner. 2012, 92, 70-80.) Reduced temperatures (e.g., 280° F.) are possible if a vacuum cooker is used. Once removed from heat and cooled the syrup hardens forming a hard, amorphous structure with a water content generally less than 5% that is referred to as hard glass candy. In the current invention, short-chain fructooligosaccharides and D-allulose replace sucrose and resistant maltodextrin replaces corn syrup.

The following examples illustrate specific embodiments and/or features of the present disclosure. The examples are given solely for the purpose of illustration and are not to be construed as limitations of the present disclosure, as many variations thereof are possible without departing from the spirit and scope of the disclosure.

### EXAMPLE 1

We attempted to prepare a hard lozenge using Nutraflora P-95, Fibersol-2, and water. One hundred and fifty-nine grams of Nutraflora P-95, 169 grams of Fibersol-2, and 122 grams of water were combined. Based on specifications provided by the ingredient suppliers, both Nutraflora P-95 and Fibersol-2 will provide 145.5 grams of dietary fiber. As a result, both Nutraflora P-95 and Fibersol-2 each contribute 50% of the dietary fiber in the final product. The combined material was heated with a goal to reach 300° F. in order to produce an amorphous product with a moisture content of less than 5% which is indicative of a hard glass candy structure. However, we were only able to reach a temperature of 260° F. before the syrup became so thick that it was impractical to produce lozenges. Moisture content of the sample using the Karl Fischer method (AOAC Ca 2e-84) was found to be 8.97% which is much higher than the 5% target that is anticipated for a hard glass candy structure. This formulation is not practical for the commercial production of a high-fiber hard lozenge.

As a follow-up, the formulation was modified to include D-allulose in an effort to produce a commercially viable high-fiber hard lozenge. Hard lozenges were prepared using Nutraflora P-95, Fibersol-2, D-allulose, and water. One hundred and fifty-nine grams of Nutraflora P-95, 169 grams of Fibersol-2, 77 grams of D-allulose, and 150 grams of water were combined and heated to 300° F. The syrup was removed from the heat, allowed to cool, and formed into hard lozenges. Based on specifications provided by the ingredient suppliers, both Nutraflora P-95 and Fibersol-2 will provide 145.5 grams of dietary fiber. As a result, both Nutraflora P-95 and Fibersol-2 each contribute 50% of the dietary fiber in the final product. Also, based on ingredient specifications provided by the supplier, the final product would contain approximately 75% dietary fiber on a dry matter basis. Moisture content of the sample using the Karl Fischer method (AOAC Ca 2e-84) was found to be 3.58% and aligns with the target of less than 5% typical of hard glass candy.

### EXAMPLE 2

Hard lozenges were prepared using Nutraflora P-95, Fibersol-2, D-allulose, and water. Two thousand nine hundred and two grams of Nutraflora P-95, 1,533 grams of Fibersol-2, 1,040 grams of D-allulose, and 2,026 grams of water were combined and heated to dissolve the ingredients and create a syrup. The resulting syrup was cooled and placed in a 10-liter plastic jug. The 10-liter plastic jug was chilled to 38° F. and then placed in an insulated shipping container with refrigerant packs and sent to the University of Wisconsin for further processing. Using a vacuum kettle, the batch was heated to 280° F. then the vacuum was taken to 20″ Hg and held for 4 minutes. The material was cooled, formed into a rope, and lozenges prepared using a drop roller. Based on specifications provided by the ingredient suppliers, Nutraflora P-95 contributes 67% of the dietary fiber while Fibersol-2 contributes 33% of the dietary fiber to the final product. A sample was subjected to X-ray diffraction (XRD) to determine the crystallinity of the hard lozenge by H&M Analytical Services, Inc., 18C Hornerstown Rd, Cream Ridge, N.J. 08514. The lozenge was ground to a fine powder with a mortar and pestle. The powder was backloaded into a standard sample cup and put into a Panalytical X′Pert Pro diffractometer using Cu radiation at 45 KV/40 mA. The scan was run over the 2θ range of 6° to 80° with a step size of 0.0167° and an accumulated counting time of 250 seconds per step. Based on the pattern collected, no crystalline peaks were noted and the sample was 100% amorphous. An amorphous structure indicates that a hard glass candy structure was obtained. Free sugars (fructose, glucose, lactose, maltose, sucrose) were also measured in the sample using a modification of AOAC method 982.14 to determine if the Nutraflora P-95 or Fibersol-2 were degraded during processing. Based on ingredient specifications for sugar content of Nutraflora P-95 and Fibersol, the hard lozenge may contain up to 5% by weight total sugars. Analysis of the sample showed that it contained 4.84% total sugars indicating that the primary ingredients, Nutraflora P-95 and Fibersol-2, were not degraded during thermal processing. Moisture content of the sample using the Karl Fischer method (AOCS Ca 2e-84) was found to be 1.76%. Sugar and moisture analyses were performed by Eurofins Scientific Inc., 2200 Rittenhouse Street, Suite 50, Des Moines, Iowa 50321.

### EXAMPLE 3

Hard lozenges were prepared using Nutraflora P-95, Fibersol-2, D-allulose, water, peppermint oil (www.LorAnnOils.com), and stevia extract (Shandong Haigen Biotechnology). Two hundred and twelve grams of Nutraflora P-95, 112 grams of Fibersol-2, 76 grams of D-allulose, and 149 grams of water were combined and heated to 300° F. The syrup was removed from the heat and one gram of stevia extract and 1.2 milliliters of peppermint oil were added. After mixing, the syrup was allowed to cool and formed into hard lozenges. Based on specifications provided by the ingredient suppliers, Nutraflora P-95 will provide 194 grams of dietary fiber while Fibersol-2 will provide 97 grams of dietary fiber. As a result, Nutraflora P-95 contributes 67% of the dietary fiber while Fibersol-2 contributes 33% of the dietary fiber to the final product. Free sugars (fructose, glucose, lactose, maltose, sucrose) were also measured in the peppermint flavored sample using a modification of AOAC method 982.14 to determine if the Nutraflora P-95 or Fibersol 2 were degraded during processing. Based on ingredient specifications for sugar content of Nutraflora P-95 and Fibersol, the hard lozenge may contain up to 5% by weight total sugars. Analysis of the sample showed that it contained 4.57% total sugars indicating that the primary ingredients, Nutraflora P-95 and Fibersol-2, were not degraded during thermal processing. Moisture content of the sample using the Karl Fischer method (AOCS Ca 2e-84) was found to be 3.63%. Sugar and moisture analyses were performed by Eurofins Scientific Inc., 2200 Rittenhouse Street, Suite 50, Des Moines, Iowa 50321.

1. A composition comprising:
a nondigestible fructooligosaccharide, a nondigestible glucose polymer, and a nonnutritive bulk sweetener in a hard, amorphous structure with a water content less than 6 wt %;
wherein said nondigestible fructooligosaccharides and said nondigestible glucose polymers account for 70 to 79 wt % on a dry matter basis; and
sucrose, glucose, and fructose account for less than 5 wt % on a dry matter basis.

2. A composition as in claim 1 wherein said nondigestible fructooligosaccharides contribute 50 to 75 wt % and preferably 67 wt % of the dietary fiber and said nondigestible glucose polymers contribute 25 to 50 wt % and preferably 33 wt % of the dietary fiber.

3. A composition as in claim 1 wherein said nondigestible fructooligosaccharide comprises 1-kestose, nystose, and β-fructofuranosylnystose, said nondigestible glucose polymer is resistant maltodextrin, and said nonnutritive bulk sweetener is D-allulose.

4. A composition comprising:
a 1-kestose, nystose, and β-fructofuranosylnystose, a resistant maltodextrin, and D-allulose in a hard, amorphous structure with a water content less than 6 wt %;
wherein said 1-kestose, nystose, and β-fructofuranosylnystose and said resistant maltodextrin account for 70 to 79 wt % on a dry matter basis; and
sucrose, glucose, and fructose account for less than 5 wt % on a dry matter basis.

5. A composition as in claim 4 wherein said 1-kestose, nystose, and β-fructofuranosylnystose contribute 50 to 75 wt % and preferably 67 wt % of the dietary fiber and said resistant maltodextrin contributes 25 to 50 wt % and preferably 33 wt % of the dietary fiber.

6. A method of delivering dietary fiber, comprising:
a nondigestible fructooligosaccharide, a nondigestible glucose polymer, and a nonnutritive bulk sweetener in a hard, amorphous structure with a water content less than 6 wt % that dissolves in the mouth;
wherein said nondigestible fructooligosaccharide and said nondigestible glucose polymer account for 70 to 79 wt % on a dry matter basis; and
sucrose, glucose, and fructose account for less than 5 wt % on a dry matter basis.

7. A method as in claim 6 wherein said nondigestible fructooligosaccharide contributes 50 to 75 wt % and preferably 67 wt % of the dietary fiber and said nondigestible glucose polymer contributes 25 to 50 wt % and preferably 33 wt % of the dietary fiber.

8. A method as in claim 6 wherein said nondigestible fructooligosaccharide comprises 1-kestose, nystose, and β-fructofuranosylnystose, said nondigestible glucose polymer is resistant maltodextrin, and said nonnutritive bulk sweetener is D-allulose.

9. A method of delivering dietary fiber, comprising:
a 1-kestose, nystose, and β-fructofuranosylnystose, a resistant maltodextrin, and D-allulose in a hard, amorphous structure with a water content less than 6 wt %;
wherein said 1-kestose, nystose, and β-fructofuranosylnystose and said resistant maltodextrin account for 70 to 79 wt % on a dry matter basis; and
sucrose, glucose, and fructose account for less than 5 wt % on a dry matter basis.

10. A method as in claim 9 wherein said 1-kestose, nystose, and β-fructofuranosylnystose contribute 50 to 75 wt % and preferably 67 wt % of the dietary fiber and said resistant maltodextrin contributes 25 to 50 wt % and preferably 33 wt % of the dietary fiber.
